(19) United States
(12) Patent Application Publication
(10) Pub. No.: US 2009/0325918 A1
(43) Pub. Date: Dec. 31, 2009

(54) Synthesis and separation of optically active isomers and cyclopropyl derivatives of spironolactone and their biological action

(76) Inventors: John C. Somberg, (US); Vassant V. Ranade, (US)

Correspondence Address: Gildo E. Fato, Esq.;c/o Laurence M. Dunlap, 14047 Petronella Dr., Libertyville, IL, 60048, US

(21) Appl. No.: 12/214,352

(22) Filed: Jun. 16, 2008

(51) Int. Cl. A61K 31/585; A61K 31/56; C07J 71/00; C07J 31/00; A61P 9/00; A61P 9/12; A61P 9/04

(52) U.S. Cl. 514/173; 514/177; 540/23; 552/523

(57) ABSTRACT

Methods for separation and synthesis of the optically active 7-thioester isomers and mono or bis-cyclopropyl derivatives of spironolactone are provided. Preferred stereoisomerically purified 7-thioester isomers and mono or bis-cyclopropyl derivatives of spironolactone have fewer effects mediated by gonadal steroid receptors relative to effect mediated by minteralocorticoid progesterone receptors, compared to the stereoisomerically unpurified form of the compound. These optically active compounds can be useful for obtaining reduction in moderate essential hypertension and it the treatment of congestive heart failure in humans with minimized undesirable side effects such as gynecomastia, tender breast enlargement and menstrual irregularities in women, and loss of libido in men.

### BACKGROUND OF THE INVENTION

Spironolactone (SL) is known to be a potent aldosterone antagonist at mineralocorticoid steroid hormone receptors, and it is widely used in humans for the treatment of essential hypertension, congestive heat failure and refractory edema or hyperaldosteronism. However, the prolonged use of SL is associated with undesirable endocrine side effects such as gynecomastia and lose of libido in men and menstrual irregularities in women due to interaction of SL with gonadal steroid hormone biosynthesis and target cell gonadal steroid receptors.

The nature and prevalence of the undesirable side effects limit the usefulness of spironolactone as a therapeutic agent. Gynecomastia or tender breast enlargement has been found to occur in 10% of hypertensive patients using spironolactone for therapy as compared to 1% of men in the placebo group. Recent studies by Pitt, et al. with spironolactone have shown that in patients with congestive heart failure (CHF) taking digoxin and a loop diuretic—spironolactone therapy in conjunction with digitalis and ACE inhibitor—reduces mortality by 30%. See Pitt, B., et al., The Effect of Spironolactone on Morbidity and Mortality in Patients with Severe Heart Failure, Randomized Aldactone Evaluation Study Investigors; N. Engl. J. Med., 1999, 341:709-717. These authors stated that the 30% reduction in the risk of death among patients in the group receiving spironolactone could be attributed to a lower risk of both death from progressive heart failure and sudden death from cardiac arrhythmic causes. In addition, they found that the frequency of hospitalization for worsening heart failure is 35% lower in the spironolacotone treated group than in the placebo group. These authors concluded that patients who received spironolactone had a significant improvement in the symptoms of severe heart failure caused by systolic left ventricular dysfunction. Overall, 8% of the patients in the spironolactone group discontinued treatment because of adverse events. The purpose of the present invention is to make available the individual chiral isomers of spironolactone that would be effective in treating CHF and in reducing hypertension, and at the same time would be devoid of undesirable side effects such as gynecomastia, lose of libido in men, and menstrual irregularities in women.

Spironolactone is the name commonly used for a specific spirolactone that has the full chemical name 17-hydroxy-7-alpha-mercapto-3-oxo-17-alpha-pregn-4-ene-21-carboxylic acid gamma-lactone acetate. The term “spirolactone” denotes that a lactone 10 ring (i.e., a cyclic ester) is attached to another ring structure in a spiro configuration (i.e., the lactone ring shares a single carbon atom with the other ring). Spirolactones that are coupled to steroids are the most important class of spirolactones from a pharmaceutical perspective, so they are widely referred to in the pharmaceutical arts simply as spirolactones. As used herein, “spironolactone” refers to a molecule comprising a lactone structure coupled via a spiro configuration to a steroid structure or steroid derivative.

Spironolactone, its activities, and modes of synthesis and purification are described in a number of U.S. patents, notably U.S. Pat. Nos. 3,013,012, 4,529,811 and 4,603,128.

Intracellular receptors (IRs) form a class of structurally-related genetic regulators that act as ligand-dependent transcription factors. See Evans, R. M., “The Steroid and Thyroid Hormone Receptor Superfamily”, Science, May 13, 1988; 240(4854):889-95. Steroid receptors are a recognized subset of the IRs, including the progesterone receptor (PR), androgen receptor (AR), estrogen receptor (ER), which can be referred to collectively as the gonadal steroid receptors, glucocorticoid receptor (GR), and mineralocorticoid receptor (MR). Regulation of a gene by such factors requires both the IR itself and a corresponding ligand that has the ability to selectively bind to the IR in a way that affects gene transcription.

Ligands for the IRs can include low molecular weight native molecules, such as the hormones aldosterone, progesterone, estrogen and testosterone, as well as synthetic derivative compounds such as medroxyprogesterone acetate, diethylstilbesterol and 19-nortestosterone. These ligands, when present the fluid surrounding a cell, pass through the outer cell membrane by passive diffusion and bind to specific IR proteins to create a ligand/receptor complex. This complex then translocates to the cell's nucleus, where it binds to a specific gene or genes present in the cell's DNA. Once bound to DNA, the complex modulates the production of the protein encoded by that gene. In this regard, a compound that binds to an IR and mimics the effect of the native ligand is referred to as an “agonist”, while a compound that binds to an IR and inhibits the effect of the native ligand is called an “antagonist”.

The therapeutic mechanism of action of spironolactone involves binding to intracellular mineralocorticoid receptors (MRs) in kidney epithelial cells, thereby inhibiting the binding of aldosterone. Spironolactone has been found to counteract the sodium reabsorption and potassium excretion effects of aldosterone and other mineralocorticoids. Spironolactone has also been shown to interfere with testosterone biosynthesis, has anti-androgen action and inhibits adrenal aldosterone biosynthesis. Large doses of spironolactone in children appear to decrease the testosterone production rate.

Spironolactone is found to exhibit intra-individual variability of pharmacokinetic parameters and it presumably belongs to the group of drugs with high inter-subject variability. Spironolactone has poor water solubility and dissolution rate.

In order to prolong the half-life and decrease the side effects associated with spironolactone, syntheses of spironolactone derivatives have been developed (e.g. synthesis of mexrenone, prorenone, spirorenone). Slight modifications of the spironolactone steroid skeleton, e.g. such as formation of 11β-allenic and epoxy compounds, have been shown to effect important variations in the affinity and specificity for the mineralocorticoid receptor. These results suggest that it is possible to develop spironolactone analogues that do not interact with the androgen receptor or cytochrome P-450 and are therefore free of spironolactone undesirable side-effects.

### BRIEF DESCRIPTION OF THE DRAWINGS

FIG. 1 illustrates mineralocorticoid antagonists.

FIG. 2 illustrates the major pathways of spironolactone metabolism.

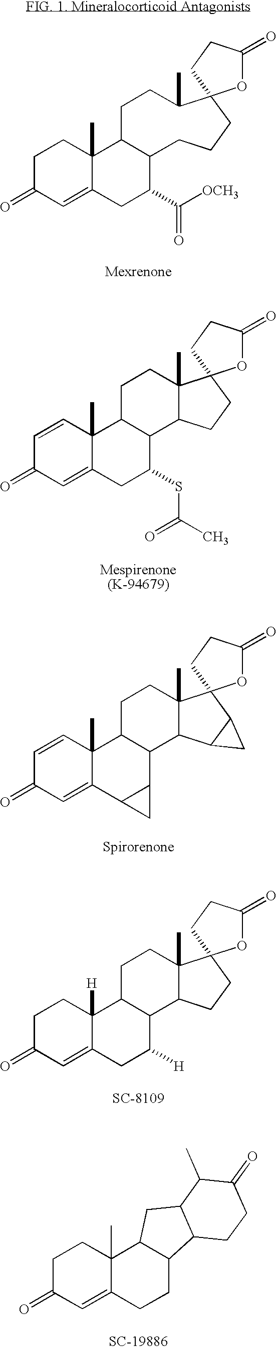

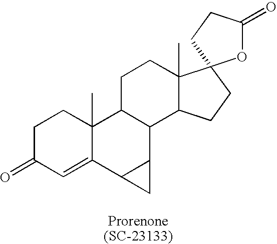

The pharmacology of spironolactone, mineralocorticoids, glucocorticoids and their corresponding anti-compounds have been reviewed extensively by several workers (Saunders and Alberti, et al.). The site of action of spironolactone is believed to be at the epithelium of the distal nephron where it blocks the anti-natriuretic and the kaliuretic activity of aldosterone. The diuretic action of spironolactone is linked to its antagonism of aldosterone at the cytoplasmic mineralocorticoid receptor. Spironolactone also blocks further promotion of the physiological response to aldosterone by maintaining the receptor in a conformation that has a low affinity for the critical hormone/receptor chromatin target sites. In vitro investigation with renal cytoplasmic mineralocorticoid receptors has been use to test the changes in the 3-dimensional molecular structure of the receptor when spironolactone or other antimineralocorticoid steroids are bound to it. These findings have facilitated the design and synthesis of new specific and potent aldosterone antagonist.

During the past several years the syntheses of new steroidal aldosterone antagonist has been developed. These investigations are based on the structures of metabolites of SL. Metabolism of SL is a very rapid and very little of the parent compound is detectable in blood or urine of humans. Consequently, the actions of SL have been attributed largely to metabolites of the drug.

The biotransformation of spironolactone in very complex including both pass I and pass II reactions. Spironolactone is well absorbed after oral dosing, but it is extensively metabolized to yield either sulfur-free (e.g. canrenone, K-canrenoate and 6,7 diols) or sulfur-containing (7 alpha-thiomethylspirolactone) metabolites (see FIG. 2). In particular, the metabolism of spironolactone begins with hydrolysis of the thioacetate group to form a 7 alpha-thiospironolactone. The intermediate 7 alpha-thiospironolactone can them either be methylated to afford the 7 alpha-thiomethyl compound and other sulfur-containing metabolites or the 7 alpha-thio group is eliminated to form canrenone, which can be further metabolized to generate several sulfur-free metabolites, some of which (e.g. canrenone) were found to be competitive inhibitors of mineralocorticoids.

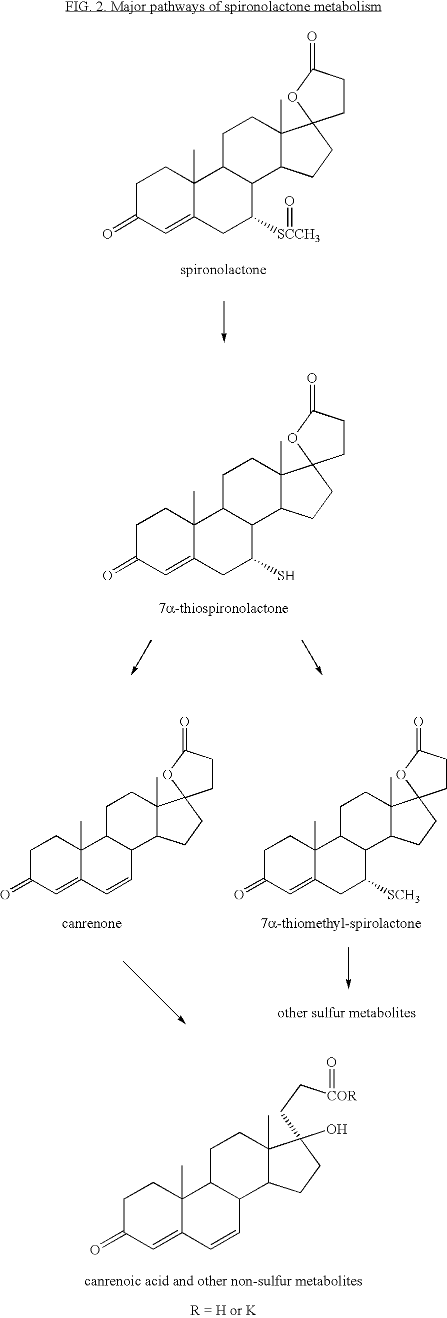

The chemistry of the spironolactone derivatives is well documented and the desired spironolactone isomer can be obtained effectively in reasonable yields. In these methods, the 7-beta is separated from the 7-alpha. The spirolactone portion is always of the singular configuration. Methods used for chiral separation of the SL isomer have also been documented.

The present invention also provide assays for biological activity (antimineralocorticoid antiglucocorticoid, for example) determination as well as clinical assays of determining the quantities of SL derivative present in the biological fluids.

### SUMMARY OF THE INVENTION

The present invention provides methods for purifying, identifying and using optically active 7-alpha or 7-beta isomers of spironolactone and spironolactone derivatives as well as compositions comprising such optically active isomers. Such optically active isomers having desired actions mediated by mineralocorticoid receptors substantially separable from undesirable effects mediated by gonadal steroid receptors can be useful for more effective therapy of hypertension, congestive heart failure, refractory edema, hyperaldosteronism, or cardiac fibrosis. Also disclosed are methods for assaying the levels of such isomers present in the biological fluids.

### DETAILED DESCRIPTION OF THE INVENTION

This invention provides compounds that are optically active isomers of spironolactone and their assessment of biological activity as described. After separation, purification and characterization, the SL isomers can be tested for the criteria described.

### General Methods

### HPLC Methods of Separating Isomers

A newly developed reversed phase HPLC-method for determination of spironolactone, canrenone, and 7 alpha-thiomethylspirolactone in plasma is used in this study after partial adaptation. The method entails solid phase extraction and UV detection. Briefly, after addition of the internal standard, an aliquot of the ample is added to a Bond-Elut C2 cartridge. The cartridge is washed with water and 30% methanol and eluted with acetonitrile. After evaporating to dryness under a gentle stream of nitrogen, the residue is reconstituted in 250 μl of tetrahydrofuran/water (30/70 v/v, pH 3.0) and 100 μl is injected onto the HPLC system. The system consists of mobile phases (A: tetrahydrofuran/purified water 30/70 v/v; B: acetonitrile/purified water 10/90 v/v), the analytical column Hypersil phenyl, 5 μm, 250×4.6 mm) and a precolumn (Spherisorb ODS II, 10 μm, 40-4.6 mm). The flow rate is set to 1.0 ml/min on both pumps. The valve switching is performed after about 5 minutes in “load” position and 5-30 minutes in “elute” position. Peaks are monitored by UV detection on 2 different wave lengths (240 nm for spironolactone and 7 alpha-thiomethylspirolactone; 240 nm for canrenone).

LiChrosorb RP-18 and RP-8 columns (Chrompack, Middleburg, The Netherlands) of the earn dimensions are used. The chromatographic conditions that are used are listed below. The eluents are prepared by mixing the components in the stated ratios (v/v).

### Chemicals And Reagents

All reagents are of analytical grade. Acetonitrile is HPLC grade. The diuretics are obtained from commercial suppliers, such as Alltech Associates of Deerfield, Ill.; Sigma Chemical Company, St. Louis, Mo.; and Searle Laboratories, Skokie, Ill.

High-Performance Liquid Chromatographic Conditions

| | |
|---|---|
| Column | LiChrosorb RP-8, 5 μm. 150 × 4.6 mm I.D. |
| Eluent | Acetonitrile-0.05 M phosphate buffer, pH 4 (45:55) |
| Flow-rate | 1 ml/min |
| Temperature | 25° C. |
| Detector | UV detector, wavelength 286 nm or 271 nm |
| Recorder | Chart speed 0.5 cm/min |
| Sample loop | 10 μl |

The concentration of canrenone is determined in plasma and urine samples by high-performance liquid chromatography (HPLC) with UV-detection. An aliquot of 300 ng of spironolactone derivative is added to the samples as internal standard, which are then extracted twice with 1 ml n-hexane-toluene (1:1, v/v). The organic phase is taken to dryness and re-dissolved in 250 μl HPLC eluent (methanol-water, 60:40, v/v). (25×4.6 mm; 5 μm). Detection is performed with the UV detector set at λ=285 nm.

### Flurometric Method

Five ml of water is a reagent blank and 5 ml of working standards containing 0.05 μg and 0.20 μg of SC-9376 are carried through the entire procedure. Lower sales are read vs. the 0.05 μg standard at full scale, and higher samples vs. the 0.20 μg standard. Fluorescence readings are proportional to the concentrations of the standards in this range.

Pipette 0.2 ml of heparinized plasma into a 50-ml polyethylene-stoppered centrifuge tube, dilute to 5 ml with water and add 15 ml of methylene chloride (Du Pont refrigeration grade, redistilled). Shake for 30 seconds, centrifuge and discard the aqueous supernatant. Add 1 ml 0.1 N NaOH, shake 15 seconds, centrifuge and discard the supernatant. Transfer a 10-ml aliquot of the methylene chloride phase to another tube containing 2 ml of 65% aqueous sulfuric acid, shake 30 seconds, centrifuge and remove organic phase by aspiration. The material is allowed to stand at room temperature for about 1 hour and then about 1 ml of the sulfuric acid phase in transferred to a quartz cuvette. Fluorescence intensity is determined in an Aminco-Bowman spectrophotofluorometer (activation maximum, 465 nm).

### Gas Liquid Chromatography

The GLC estimation is carried out on a Fractovap Model 251 series 2150 (Carlo Erba) instrument equipped with a Nickel-63 electron capture detector. A 6-foot, 0.4 mm internal diameter, U-shaped glass column, packed with OV-17 2% or XE-60 1% on gas chrom A, 100-120 mesh (Applied Science Lab) is conditioned for 3 days before use. Argon with 10% methane which passed through a molecular sieve before entering the column is used as the carrier gas. The conditions of analysis are: column 255° C., detector 275° C., carrier gas flow 30 ml/min. Samples are injected on the column with a 10 μl Hamilton syringe. The injector in not heated.

### EXAMPLE 1

### Chiral Separation

The separation of 7 beta isomer of SL is schematically described below.

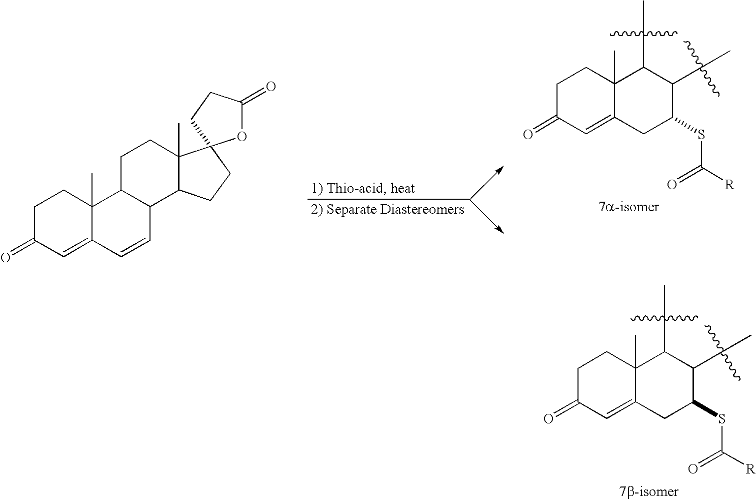

### Chromatographic Method for Isolation of SL Isomers

The basic method is described in Chan, Ky, et al., J. Chromatog, Nov. 15, 1991:571 (1-2) 291-297. The separation is performed using spectra-physics HPLC instrument and UV variable wavelength detector set at 254 nm. For chiral separation, the chromatographic column is either a pre-packed 25 mm×4.6 mm ID Cyclobond 1 (5 μm particle size), or a pre-packed 150 mm×4 mm ID Resolvosil BSA-7 column (5 μm) operated using the conditions described herein.

Analysis of the isomers present in the peaks in the chromatograms and their chiral extract purity analysis can be determined in each case by high resolution NMR spectroscopy using a chiral shift reagent. Based on this information and the determination of molecular weight by mass spectrometry and/or optical activity, structural configuration is assigned to each isomer. Eluted samples of isomers may be re-chromatographed in order to obtain adequate quantities of isomers having desired optical purity for study. For future use, reference standards that are optically pure will be compared for confirmation of purity and identity to the isolated isomers that are obtained after their chromatographic separation.

### EXAMPLE 2

### Chemical Synthesis of Optical Isomers

As an example, the desire spironolactone 7-beta-isomer is synthesized following the scheme that is described below:

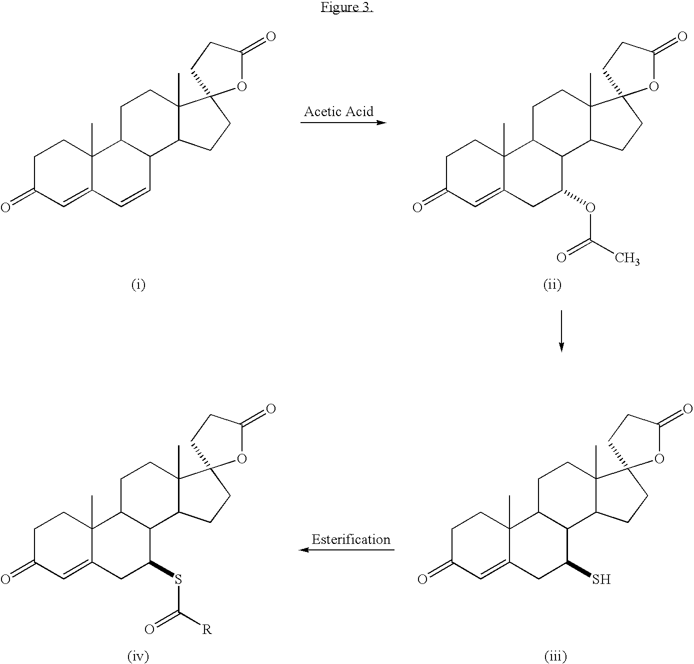

Diene (i) is prepared from commercially available starting materials using methods well known in the art of chemical synthesis.

Diene (i) is treated with acetic acid and the mixture is heated to reflux to yield 7-alpha-acetate ester (ii). The 7-alpha-ester (ii) is further subjected to nucleophilic substitution, followed by hydrolysis to obtain the 7-beta-isomer (iii). The 7-beta-isomer (iii) is then esterified with an acyl halide in the presence of a base to generate the desired spironolactone 7-beta-isomer (iv).

### EXAMPLE 3

### Preparation of Radiolabeled Probe Compounds of the Invention

Using known methods, the compounds of the invention may be prepared as radiolabeled probes by carrying out their synthesis using precursors comprising at least one atom that is a radioisotope. The radioisotope is preferably selected from at least one of carbon (preferably ¹⁴C), hydrogen (preferably ³H), sulfur (preferably ³⁵S), or iodine (preferably I). Such radiolabeled probes are conveniently synthesized by a radioisotope supplier specializing in customer synthesis of radiolabeled probe compounds. Such suppliers include Amersham Corporation, Arlington Heights, Ill.; Cambridge Isotope Laboratories, Inc., Andover, Mass.; SRI International, Menlo Park, Calif.; Wizard Laboratories, West Sacramento, Calif.; ChemSyn Laboratories, Lexena, Kans.; American Radiolabeled Chemicals, Inc., St. Louis, Mo.; and Moravek Biochemicals Inc., Brea, Calif.

Tritium labeled probe compounds are also conveniently prepared catalytically via platinum-catalyzed exchange in tritiated acetic acid, acid-catalyzed exchange in tritiated trifluoroacetic acid, or heterogeneous-catalyzed exchange with tritium gas. Tritium labeled probe compounds can also be prepared, when appropriate, by sodium borotritide reduction. Such preparations are also conveniently carried out as a custom radiolabeling by any of the suppliers listed in the preceding paragraph using the compound of the invention as substrate.

### EXAMPLE 4

### Isolation and Purification Procedure

The optical isomers of spironolactones may be isolated from fluid sample such as urine or blood as follows:

Extraction from Urine

The urine sample is extracted with dichloromethane and the extract washed with NaOH (0.1 N) and then with water to neutrality. The residue obtained after evaporation of the dichloromethane extract is purified on TLC in three different systems: benzene-acetone-water, (150:100:0.4); chloroform-ethanol, (90:10); ethyl acetate-cyclohexane-ethanol, (45:25:10), using aldosterone as reference standard.

The extract is then purified by high performance liquid chromatography (HPLC) on a Waters 6000 A, 480 U.V. detector instrument with radial pressure. The extract is first run through a C₁₈ 10μ column using methanol-water (70:30) as the eluent, followed by a silica 5μ column using dichloromethane-methanol (95:5). In both cases, the rate of the eluent is 1.5 ml/min. A small part of the extract is subjected to heptafluorobutyrylation for GLC investigation.

Extraction from Blood

Human heparinated plasma (150 ml) and serum (400 ml) are extracted with 3 volumes of dichloromethane. The dichloromethane extract is washed first with 0.1 N sodium hydrogen carbonate and then with water until the pH of the water is neutral. The dichloromethane layer is evaporated to dryness and purified by thin layer chromatography as described above.

### EXAMPLE 5

### Determination of Antialdosterone Activity in Rats

The aldosterone antagonist activity of the various optical isomers of spironolactone and derivatives can be determined in a rat model. Briefly, male Wistar rats having body weight of 140-160 g are adrenalectomized under ether anesthesia 5 days prior to the diuresis experiment. Adrenalectomized Wistar rats are injected with 1 mg/kg of fluocortolone caproate on the day of surgery and 10 mg of fluocortolone caproate subcutaneously 1 day before the diuresis test. The diuresis test is performed by infusing the subject rats intravenously with saline-glucose solution (0.05% NaCl, 5% glucose) containing aldosterone (50 μg/L) at a rate of 3 mL/h. The rats are divided into three groups of 7 to 10 rate per group, and the aldosterone antagonist to be tested is administered 1 hour before the start of the aldosterone infusion. The first group of rats is dosed orally with 6.7 mg/kg, the second group with 13.4 mg/kg and the third group with 26.8 mg/kg. As a control for antiandrogenic activity, a group of control rats is treated with testosterone propionate. Urine excretion is measured several times per hour. Sodium and potassium concentrations in urine are determined by flame photometry.

The aldosterone antagonist activity of a test compound is assessed by the ability of the compound to reverse the effect of aldosterone on the urinary Na/K ratio. The dose-response relationship for each test compound is determined by regression analysis after logarithmic transformation of the doses. The relative effect of each test compound is normalized to that of the standard substance, spironolactone, which is assigned a value of 100.

### Diuresis Protocol

For example, immediately after surgery the rate are injected with fluocortolone caproate (10 mg/kg s.c.).

The rats are again pretreated with fluorocrtolone (1.25 mg/kg s.c.) four days after adrenalectomy and before the diuresis experiment. Following adrenalectomy, the animals are given a 1% NaCl solution to drink and a standard diet, Altromin R (Altrogge, Lage/Lippe, Fed. Rep. of Germany), ad libitum. Food is withdrawn 16 hours before the beginning of the diuresis experiment. In these diuresis experiments, a continuous i.v. infusion (3 m/animal/h) of an isotonic saline or saline-glucose solution (9 g NaCl/l or 2 g NaCl/l and 43 g glucose/L or 0.5 g NaCl/L and 52 g glucose/L) is administered to the rats via a tail vein aver a period of 15 or 21 hours. With the exception of solutions administered to adrenalectomized controls, the infusion solution additionally contained d-aldosterone 150 μg/kg/h. By this means an increased constant urine flow and a constant mineralocorticoid effect proved by sodium retention, enhanced potassium excretion and hence a reduction of the Na/K-ratio is generated. The number of rats per experimental group is 7-10.

The steroid to be tested is either administered orally 1 hour prior to starting the i.v. infusion or infused intravenously throughout the course of the experiment. For oral administration microcrystalline suspensions are prepared using 0.9% saline solution with 85 mg/100 ml macrogol sterate (Myr), (Atlan-Chemie, Essen, Fed. Rep. of Germany) as vehicle. For i.v. administration, the water soluble steroids are to the infusion solution.

For the evaluation of orally active steroids, 8 or 9 groups of rats generally are considered simultaneously in each experiment:
i.v. infusion without aldosterone (adrenalectomized control);
i.v. infusion with aldosterone (aldosterone control);
i.v. infusion with aldosterone and three different oral doses of a reference spirolactone;
i.v. infusion with aldosterone and three (to four) different oral doses of a test compound.
   For the evaluation of the water-soluble steroid ZK 97872 (Δ-15β, 16β-methylene-derivative of potassium canrenoate) a similar procedure with the following modifications is used: the reference compound (potassium canrenoate) as well as the test compound (ZK 97872) are infused intravenously together with aldosterone. One hour samples of urine are collected using an automatic fraction collector. Urinary sodium and potassium concentrations are determined.

### EXAMPLE 6

### Determination of Binding to Progesterone and Androgen Receptors

To achieve initial information on the progestogenic and antiandrogenic potential of the test compounds, their affinity to binding sites specific for progestogens and androgens, respectively, is measured. Tritiated progesterone and dihydrotestosterone are used as ligands for the determination of the affinity of the test substance of the progesterone and androgen receptor isolated from the rabbit uterus and the rat prostate gland, respectively. The competition factors are defined as the multiple of the concentration to obtain displacement equivalent to the standard. A high competition factor indicates low binding strength, and a low competition factor indicates high affinity.

The procedure used has been described in detail for the determination of estrogen receptor preparations (cytosol) prepared from rabbit uterus (progesterone-receptor) and from rat prostate (androgen-receptor). ³H-Progesterone and ³H-dihydrotestosterone were used as tracers. Unlabeled progesterone and dihydrotestosterone are used as references. From the concentrations needed for each test compound to displace the tracer from its binding site to the same degree as the reference steroid, the relative binding affinity was calculated.

### EXAMPLE 7

### Determination of Antiandrogenic Activity in Rats

Castrated male rats (SPF, Wistar strain) weighing about 100 g each are treated once a day with varying doses of test compounds (1,2,4,8, or 16 mg/rat per day, sc, or 1,3,10, 30, or 100 mg/rat per day, po) in combination with 0.1 mg/rat per day (sc) of testosterone propionate (Tp) for 12 days. Control animals obtained only 0.1 mg/rat per day (sc) of Tp. One day after the last treatment (day 13), the animal are sacrificed and the fresh weights of seminal vesicles and ventral prostates are evaluated. Data obtained are subjected to regression/covariance analysis to compare the relative potencies of test compounds.

### EXAMPLE 8

### Determination of Progestational Activity (Clausberg Test)

Castrated infantile female rabbits (albino New Zealand) weighing 800-1000 g are administered a daily subcutaneous dose of 0.5 μg of estradiol for 6 days (priming). Thereafter, the animals are treated with test compounds (total dose 3,10,30, or 100 mg/rabbit, subcutaneously or 30, 100, or 300 mg/rabbit, orally) for 5 days. On day 12, the animals are sacrificed, and the uteri are removed for histological studies. The rate of the glandular development in endometrium is evaluated by using the McPhail scale (a range from 1 to 4, where 1=inactive and 4=maximal development of the glands).

### EXAMPLE 9

### Determination of Ovulation Inhibition in Rats

Mature female rats (SPF, Wistar strain) weighing about 200 g each are used in this experiment. Prior to starting the treatment, the estrous cycles of animals are controlled over a period of three cycles by means of vaginal smears. The daily treatment of animals showing a regular 4-day cycle with test compounds (30,40, or 100 mg/rat per day, orally) is initiated on the day of metestrus (day 1) and continued for 3 or 4 days. On day 4, animals are unilaterally ovariectomized, and the fallopian tube is dissected free. The tube is then microscopically examined by counting the number of ova to ascertain whether ovulation has occurred or not. Animals that have not ovulated are further treated on day 4. All animals are sacrificed on day 5, and the remaining tube is similarly examined as on day 4. The ovulation-inhibiting activity of test substances is expressed as the percent of animals in which ovulation is suppressed.

### EXAMPLE 10

### In Vitro: Binding to Renal Cytoplasmic Mineralocorticoid Receptors (MCR)

Male Sprague Dawley rats (body weight 180 g to 200 g) are adrenalectomized under phenobarbital anaesthesia 4 days prior to the MCR test, and are given 1% sodium chloride solution to drink and fed Purina rat diet. On the day of the test, 3-4 rats are anaesthetized with phenobarbital (5 mg/100 g body weight, given i.p.) and exsanguinated by the carotid artery. The kidneys are perfused with ice-cold buffer (0.17 mol/1 NaCl, 0.25 mol/1 K₂HPO₄, at pH 7.4) via vena cava; the kidneys are then removed and homogenized in Trio buffer (0.1 mol/1 Tris, 3 mmol/1 CaCl₂, at pH 7.4). The homogenate is centrifuged for 45 min at 30,000 g. The supernatant (cytosol) is taken for further incubation immediately afterwards. ³H-Aldosterone (2.5 nmol/l) and a number of unlabeled steroids in increasing concentrations are incubated together with 500 μl of cytosol for 1 hour at 23° C. This concentration of ³H-aldosterone represents the range of the Scatchard analysis, in which aldosteronerone is bound only to Type 1 binding sites.

Protein bound ³H-aldosterone is separated from the free activity by gel filtration (Biogel P-10). The activity in the protein fraction is measured in the scintillation counter after addition of 10 ml of aquasol (Tricarb Model 3385, Packard Co.).

³H-Aldosterone which is non-specifically protein bound is determined by incubating ³H-aldosterone (2.5 nmol/L) together with unlabeled aldosterone at a 1000 times higher concentration. This “non-specifically bound fraction” of the total protein bound ³H-aldosterone accounted for about 10-20% of the total and is subtracted from the total bound ³H-aldosterone.

The inhibitory effect of a non-labeled steroid on the binding of ³H-aldosterone to the receptor sites is calculated from the following relation.

$$\frac{\begin{matrix}Specific bound {}^{3} H \text{-} aldosterone \\ in presence of the substance\end{matrix} \times 100}{Specific bound {}^{3} H \text{-} aldosterone in absence of the substance}$$

The affinity for aldosterone receptors of the various substances investigated is compared with that of spironolactone in each incubation sample. The relative affinity, referred to that of spironolactone (which is taken to be 1.0) is established by the following relation:

$$\frac{Concentration of spironolactone at 50 \% displacement}{Concentration of reference substance at 50 \% displacement}$$

For each component and concentration, at least two determinations are performed in duplicate.

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications way be made therein without departing from the spirit or scope of the present invention as set forth in the claims. To particularly point out and distinctly claim the subject matter regarded as invention, the following claims conclude this specification.

What is claimed is:

1. A composition comprising of at least one stereoisomerically purified form of a compound of formula (I) or (II): 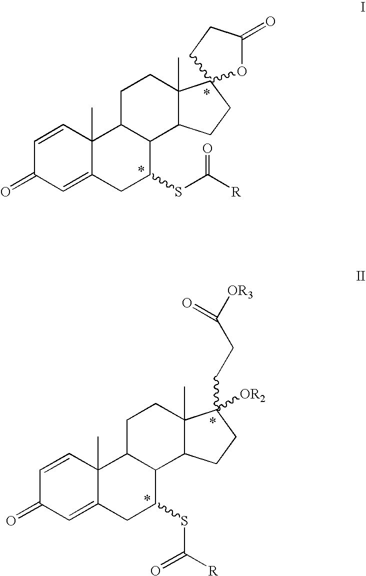
or a pharmaceutically acceptable salt thereof, wherein
R is C₁-C₆ alkyl C₂-C₆ alkyl, phenylalkyl, phenyl, wherein each phenyl group is optionally substituted with 1,2,3, or 4 substituents independently selected from the group consisting of halogen, cyano, amino, mono or dialkylamino, trifluoromethyl, trifluoromethoxy, C₁-C₆ alkyl, and C₁-C₆ alkoxy, heteroaryl, optionally substituted with 1, 2, 3, or 4 substituents independently selected from the group consisting of halogen, C₁-C₆ alkyl, C₁-C₆ alkoxy, trifluoromethyl and trifluoromethoxy, heterocycloalkyl, optionally substituted with 1,2,3, or 4 substituents independently selected from the group consisting of halogen, C₁-C₆ alkyl, C₁-C₆ alkoxy, phenyl, trifluoromethyl, and trifluoromethoxy, and —NHR² wherein
R² is selected from the group consisting of C₁-C₆ alkyl, phenylalkyl, phenyl, wherein each phenyl group is optionally substituted with 1,2,3, or 4 substituents independently selected from the group consisting of halogen, cyano, amino, mono or dialkylamino, trifluoromethyl, trifluoromethoxy, C₁-C₆ alkyl, and C₁-C₆ alkoxy, heteroaryl, optionally substituted with 1,2,3, or 4 substituents independently selected from the group consisting of halogen, C₁-C₆ alkyl, C₁-C₆ alkoxy, trifluoromethyl and trifluoromethoxy, cycloalkyl, optionally substituted with C₁-C₆ alkyl, and heterocycloalkyl, optionally substituted with 1,2,3, or 4 substituents independently selected from the group consisting of halogen, C₁-C₆ alkyl, C₁-C₆ alkoxy, phenyl, trifluoromethyl, and trifluoromethoxy;
R₃ is hydrogen, C₁-C₆ alkyl, aryl c₁-C₆ alkyl, C₁-C₆ alkoxy C₁-C₆ alkyl; heterocycloalkyl C₁-C₆ alkyl or heterocycloalkyl; and [symbol] which represents either a single or double bond, [symbol] represents either an α or β substituent, * represents a chiral center; and at least one pharmaceutically acceptable carrier, adjuvant, solvent or excipient,
wherein the stereoisomerically purified form has different effect mediated by gonadal steroid receptors relative to effect mediated by mineralocorticoid progesterone receptors and myocardial and vascular receptors involved in fibrosis, vascular necrosis and myocardial uptake of norepinephrine, compared to the stereoisomerically unpurified form of the compound.

2. A composition according to claim 1 of the formula 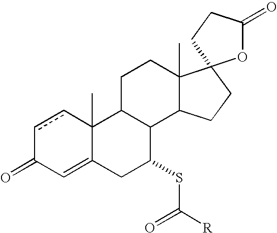
wherein R is defined as in claim 1.

3. A composition according to claim 1 of the formula 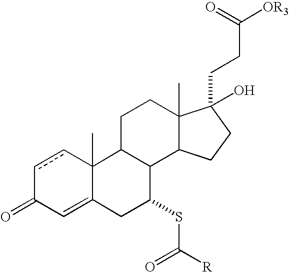
wherein R and R₃ are defined as in claim 1.

4. A composition according to claim 1 of the formula 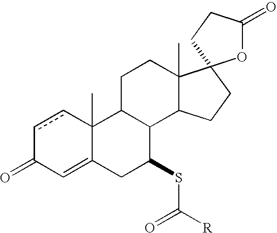
wherein R is defined as in claim 1.

5. A composition according to claim 1 of the formula 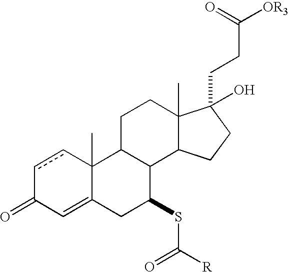
wherein R and R₃ are defined as in claim 1.

6. A composition according to claim 1 of the formula 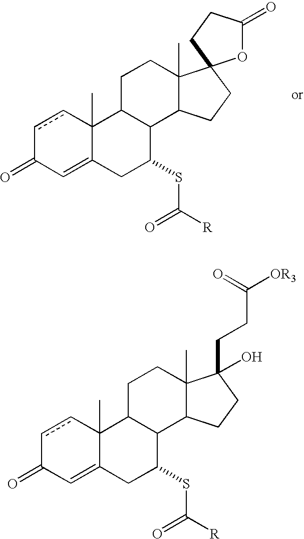
wherein R and R₃ are defined as in claim 1.

7. A composition according to claim 1 of the formula 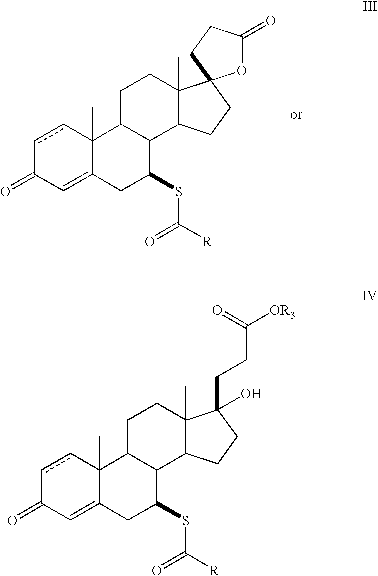
wherein R and R₃ are defined as in claim 1.

8. A compound of formula (V) or (VI) 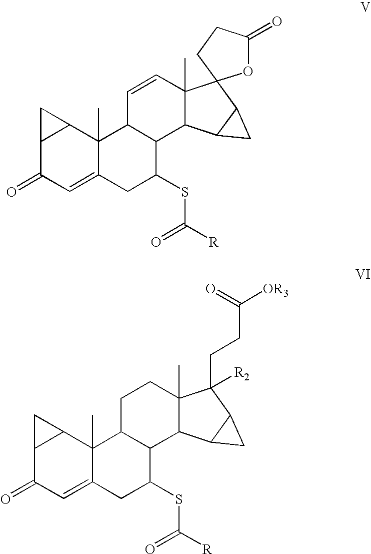
or a pharmaceutically acceptable salt thereof, wherein
R is C₁-C₆ alkyl, phenylalkyl, phenyl, wherein each phenyl group is optionally substituted with 1, 2, 3, or 4 substituents independently selected from the group consisting of halogen, cyano, amino, mono or dialkylamino, trifluoromethyl, trifluoromethoxy, C₁-C₆ alkyl, and C₁-C₆ alkoxy, heteroaryl, optionally substituted with 1, 2, 3, or 4 substituents independently selected from the group consisting of halogen, C₁-C₆ alkyl, C₁-C₆ alkoxy, trifluoromethyl and trifluoromethoxy, heterocycloalkyl, optionally substituted with 1, 2, 3, or 4 substituents independently selected from the group consisting of halogen, C₁-C₆ alkyl, C₁-C₆ alkoxy, phenyl, trifluoromethyl, and trifluoromethoxy, or —NHR² wherein
R² is selected from the group consisting of C₁-C₆ alkyl, phenylalkyl, phenyl, wherein each phenyl group is optionally substituted with 1, 2, 3, or 4 substituents independently selected from the group consisting of halogen, cyano, amino, mono or dialkylamino, trifluoromethyl, trifluoromethoxy, C₁-C₆ alkyl, and C₁-C₆ alkoxy, heteroaryl, optionally substituted with 1, 2, 3, or 4 substituents independently selected from the group consisting of halogen, C₁-C₆ alkyl, C₁-C₆ alkoxy, trifluoromethyl and trifluoromethoxy, cycloalkyl, optionally substituted with C₁-C₆ alkyl, and heterocycloalkyl, optionally substituted with 1, 2, 3, or 4 substituents independently selected from the group consisting of halogen, C₁-C₆ alkyl, C₁-C₆ alkoxy, phenyl, trifluoromethyl, and trifluoromethoxy;
R³ is hydrogen, C₁-C₆ alkyl, aryl C₁-C₆ alkyl, C₁-C₆ alkoxy C₁-C₆ alkyl; heterocycloalkyl C₁-C₆ alkyl or heterocycloalkyl; and [symbol] which represents either a single or double bond, [symbol] represents either an α or β substituent, * represents a chiral center; and at least one pharmaceutically acceptable carrier, adjuvant, solvent or excipient,
wherein the stereoisomerically purified form has different effects mediated by gonadal steroid receptors relative to effects mediated by mineralocorticoid progesterone receptors and myocardial and vascular receptors involved in fibrosis, vascular necrosis and myocardial uptake of norepinephrine, compared to the stereoisomerically unpurified form of the compound.

9. A compound according to claim 8 comprising at least one stereoisomerically purified form of a compound of the formula 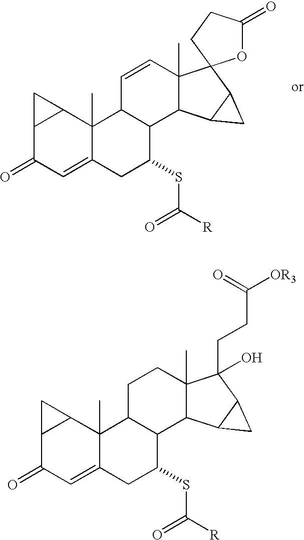
wherein R and R₃ are defined as in claim 8.

10. A compound according to claim 8 comprising at least one stereoisomerically purified form of a compound of the formula 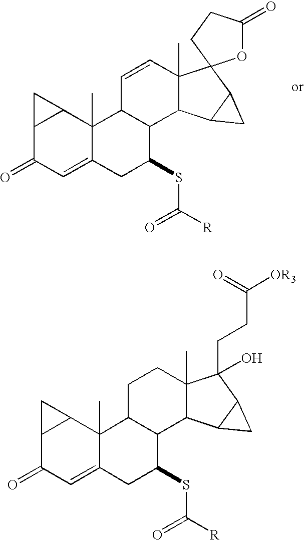
wherein R and R₃ are defined as in claim 8.

11. A compound according to claim 8 comprising at least one stereoisomerically purified form of a compound of the formula 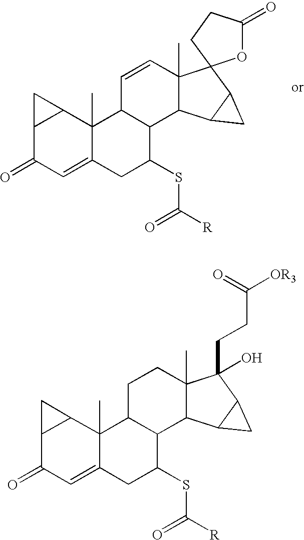
wherein R and R₃ are defined as in claim 8.

12. A compound according to claim 8 comprising at least one stereoisomerically purified form of a compound of the formula 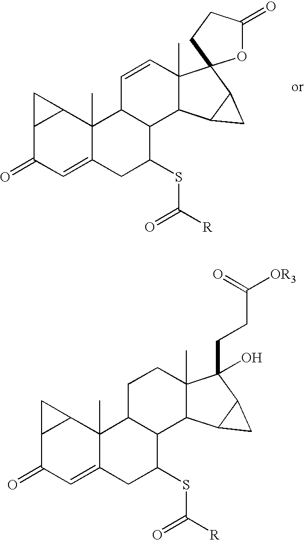
wherein R and R₃ are defined as in claim 8.

13. A compound according to claim 8 comprising at least one stereoisomerically purified form of a compound of the formula 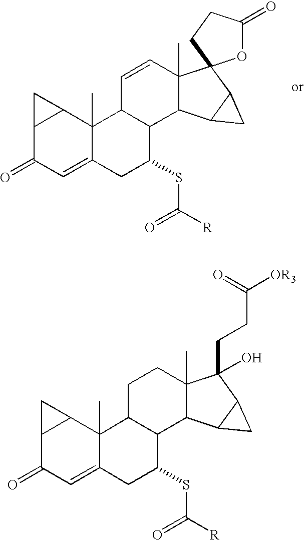
wherein R and R₃ are defined as in claim 8.

14. A compound according to claim 8 comprising at least one stereoisomerically purified form of a compound of the formula 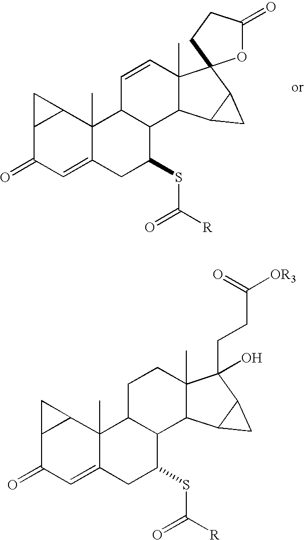
wherein R and R₃ are defined as in claim 8.

15. A compound according to claim 8 comprising at least one stereoisomerically purified form of a compound of the formula 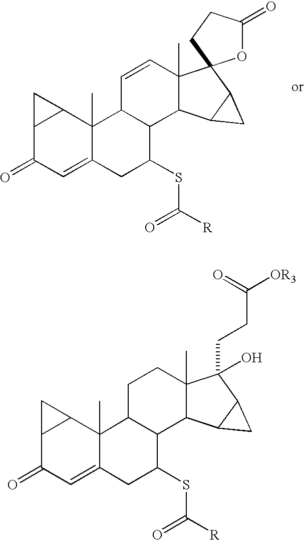
wherein R and R₃ are defined as in claim 8.

16. A compound according to claim 8 comprising at least one stereoisomerically purified form of a compound of the formula 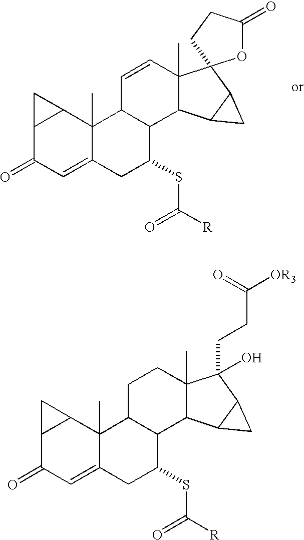
wherein R and R₃ are defined as in claim 8.

17. A packaged pharmaceutical composition comprising the pharmaceutical composition of claim 1 in a container and instructions for using the composition to treat a patient suffering from a disorder responsive to agonism, inverse agonism or antagonism of the aldosterone receptor in the kidney, myocardium or vasculature.

18. A packaged pharmaceutical composition comprising the pharmaceutical composition of claim 8 in a container and instructions for using the composition to treat a patient suffering from a disorder responsive to agonism, inverse agonism or antagonism of the aldosterone receptor in the kidney, myocardium or vasculature.

19. The packaged pharmaceutical composition of claim 17, wherein said patient is suffering from hypertension, congestive heart failure, refractory edema, hyperaldosteronism, or cardiac fibrosis.

20. The packaged pharmaceutical composition of claim 18, wherein said patient is suffering from hypertension, congestive heart failure, refractory edema, hyperaldosteronism, or cardiac fibrosis.

21. A method for the treatment of hypertension, said method comprising administering to a patient in need of such treatment or prevention a therapeutically effective amount of a compound according to claim 1, wherein the stereoisomerically purified form has less antiandrogenic effect compared to the stereoisomerically unpurified form of the compound.

22. A method for the treatment of hypertension, said method comprising administering to a patient in need of such treatment or prevention a therapeutically effective amount of a compound according to claim 8, wherein the stereoisomerically purified form has less antiandrogenic effect compared to the stereoisomerically unpurified form of the compound.

23. A method for the treatment of congestive heart failure, said method comprising administering to a patient in need of such treatment or prevention a therapeutically effective amount of a compound according to claim 1, wherein the stereoisomerically purified form has less antiandrogenic effect compared to the stereoisomerically unpurified form of the compound.

24. A method for the treatment of congestive heart failure, said method comprising administering to a patient in need of such treatment or prevention a therapeutically effective amount of a compound according to claim 8, wherein said method produces less gynecomastia or less sexual disturbances and tender breast enlargement in comparison to treatment with the stereoisomerically unpurified form of the compound.

25. A method for the treatment of refractory edema, said method comprising administering to a patient in need of such treatment or prevention a therapeutically effective amount of a compound according to claim 1, wherein said method produces less gynecomastia or less sexual disturbances and tender breast enlargement in comparison to treatment with the stereoisomerically unpurified form of the compound.

26. A method for the treatment of refractory edema, said method comprising administering to a patient in need of such treatment or prevention a therapeutically effective amount of a compound according to claim 8, wherein said method produces less gynecomastia or less sexual disturbances and tender breast enlargement in comparison to treatment with the stereoisomerically unpurified form of the compound.

27. A method for the treatment of hyperaldosteronism, said method comprising administering to a patient in need of such treatment or prevention a therapeutically effective amount of a compound according to claim 1, wherein said method produces less gynecomastia or less sexual disturbances and tender breast enlargement in comparison to treatment with the stereoisomerically unpurified form of the compound.

28. A method for the treatment of hyperaldosteronism, said method comprising administering to a patient in need of such treatment or prevention a therapeutically effective amount of a compound according to claim 8, wherein said method produces less gynecomastia or less sexual disturbances and tender breast enlargement in comparison to treatment with the stereoisomerically unpurified form of the compound.

29. A method for the treatment of cardiac fibrosis, said method comprising administering to a patient in need of such treatment or prevention a therapeutically effective amount of a compound according to claim 1, wherein said method produces less gynecomastia or less sexual disturbances and tender breast enlargement in comparison to treatment with the stereoisomerically unpurified form of the compound.

30. A method for the treatment of cardiac fibrosis, said method comprising administering to a patient in need of such treatment or prevention a therapeutically effective amount of a compound according to claim 8, wherein said method produces less gynecomastia or less sexual disturbances and tender breast enlargement in comparison to treatment with the stereoisomerically unpurified form of the compound.

31. A composition according to claim 1 wherein R³ is hydrogen, heterocycloalkyl or heterocycloalkylalkyl; and [symbol] represents a single or a double bond.

32. A composition according to claim 8 wherein R³ is hydrogen, heterocycloalkyl or heterocycloalkylalkyl; and [symbol] represents a single or a double bond.
